(19) United States
(12) Patent Application Publication
(10) Pub. No.: US 2021/0243581 A1
(43) Pub. Date: Aug. 5, 2021

(54) WIRELESS DEVICE AND WIRELESS SYSTEM

(71) Applicants: KABUSHIKI KAISHA TOSHIBA, Tokyo (JP); TOSHIBA ELECTRONIC DEVICES & STORAGE CORPORATION, Tokyo (JP)

(72) Inventor: Kiichiro OYA, Yokosuka Kanagawa (JP)

(21) Appl. No.: 16/997,755

(22) Filed: Aug. 19, 2020

(30) Foreign Application Priority Data
Feb. 4, 2020 (JP) 2020-017412

(51) Int. Cl. H04W 4/80; H04W 56/00; H04W 4/38; A61B 5/022; A61B 5/00

(52) U.S. Cl. CPC H04W 4/80; H04W 56/001; A61B 5/002; A61B 5/022; H04W 4/38

(57) ABSTRACT

A wireless device includes a clock for generating clock cycles, a wireless transmission/reception circuit, a counter configured to count the number of clock cycles output by the clock since a previous point in time, and a controller. The controller is configured to append a first counter value from the counter to transmission data. The first counter value corresponds to a time of transmission for the transmission data from the wireless transmission/reception circuit. The transmission data further includes a measurement value from a sensor device and a second counter value from the counter corresponding to a time of measurement for the measurement value.

### CROSS-REFERENCE TO RELATED APPLICATION(S)

This application is based upon and claims the benefit of priority from Japanese Patent Application No. 2020-017412, filed on Feb. 4, 2020, the entire contents of which are incorporated herein by reference.

### FIELD

Embodiments described herein relate generally to a wireless device, a wireless system, and a method of providing time-stamped data from one device to another device.

### BACKGROUND

In recent years, BLE (Bluetooth Low Energy) technology has been incorporated in various devices, for example, measuring devices that do not have a clock function, such as a sphygmomanometer and a thermometer. The measuring devices equipped with BLE may transmit measurement results to a mobile terminal, such as a smartphone or a tablet, by a BLE compatible communication.

However, when the measuring devices do not have an internal clock function, then these devices may not transmit the measurement time (time of measurement) information to the transmission destination (e.g., the mobile terminal). In such a case, users may not know the time at which the measuring devices performed their measurements. Furthermore, providing these measuring devices with a clock function, so these measuring devices may transmit time of measurement information to the transmission destination, would generally increase manufacturing costs of these devices.

### DESCRIPTION OF THE DRAWINGS

FIG. 1 is a block diagram of a wireless system including a wireless device according to a first embodiment.

FIG. 2 is a timing chart illustrating an operation at the time of measurement by a measuring device and data transmission by a wireless IC.

FIG. 3A is a view illustrating an example of transmission data transmitted as a beacon signal.

FIG. 3B is a view illustrating another example of transmission data transmitted as a beacon signal.

FIG. 4 is a block diagram of a wireless system including a wireless device according to a second embodiment.

### DETAILED DESCRIPTION

Embodiments provide a wireless device and a wireless system capable of transmitting measurement time information along with a measurement value from a measuring device to a transmission destination even when the measuring device does not have an internal clock function.

In general, according to one embodiment, a wireless device includes a clock for generating clock cycles, a wireless transmission/reception circuit, a counter configured to count the number of clock cycles output by the clock since a previous point in time, and a controller. The controller is configured to append a first counter value from the counter to transmission data. The first counter value corresponds to a time of transmission for the transmission data from the wireless transmission/reception circuit. The transmission data further includes a measurement value from a sensor device and a second counter value from the counter corresponding to a time of measurement for the measurement value.

Hereinafter, certain example embodiments will be described with reference to the drawings.

### First Embodiment

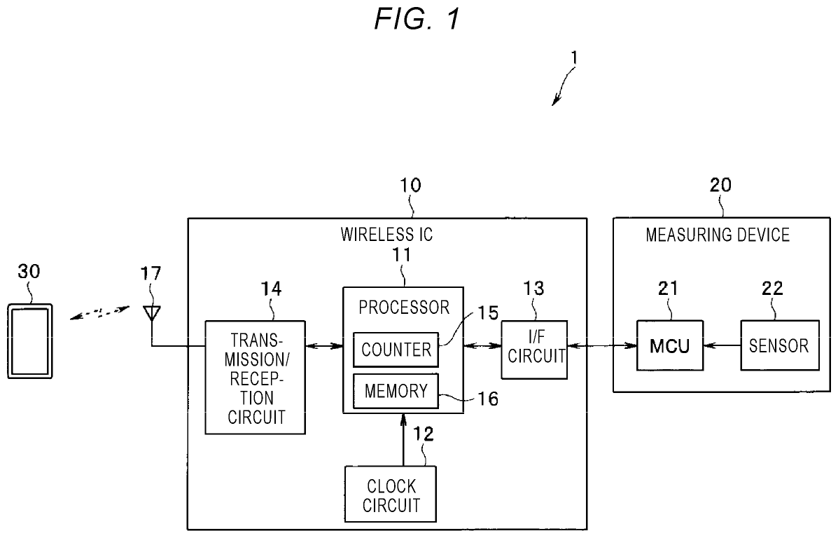

FIG. 1 is a block diagram illustrating the configuration of a wireless system including a wireless device according to a first embodiment. As illustrated in FIG. 1, the wireless system 1 includes a wireless IC 10, a measuring device 20, and a mobile terminal 30.

The wireless IC 10 is a wireless device configured as a one-chip semiconductor device, and includes a processor 11, a clock circuit 12, an interface (I/F) circuit 13, and a transmission/reception circuit 14. The processor 11 includes a counter 15 and a memory 16. The wireless IC 10 wirelessly communicates with the mobile terminal 30 via an antenna 17 using a predetermined wireless communication method (or protocol). The predetermined wireless communication method in this example is BLE communication.

Since the wireless IC 10 corresponding to the BLE communication needs to measure the transmission interval of transmission data, the clock circuit 12 is built therein. The clock circuit 12 generates a clock signal of a predetermined frequency and outputs the generated clock signal to the processor 11.

Although the clock circuit 12 may be provided inside the wireless IC 10, the clock circuit 12 is not necessarily limited thereto this configuration and may be alternatively provided outside the wireless IC 10. That is, the wireless IC 10 may have a configuration in which the clock signal is supplied from a clock circuit provided external to the wireless IC 10.

The counter 15 counts the number of clock cycles after power was initially supplied (startup) to the wireless IC 10 or the number of clock cycles after a reset signal has been received, and stores the counter value in the memory 16. The reset signal is, for example, a signal that re-initializes the state of the internal circuit of the wireless IC 10.

In this way, the wireless IC 10 according to the present embodiment may measure the counter value started from when the power was supplied to the wireless IC 10 or the counter value started from when the reset signal was input. Then, the wireless IC 10 uses the measured counter value to notify a transmission destination of the time information corresponding to when the measuring device 20 acquires the measured value.

The measuring device 20 includes a micro control unit (MCU) 21 and a sensor 22. The measuring device 20 is, for example, a sphygmomanometer that measures blood pressure, a thermometer that measures a temperature, a blood glucose meter that measures blood glucose levels, an electrocardiograph, and/or an accelerometer. The measuring device 20 may be implemented as a wearable device that acquires biological information of a measurement target at any timing, for example, by being attached to or worn on the body. When a measurement instruction is input, the MCU 21 acquires a measurement result (measurement value) from the sensor 22 and requests that the wireless IC 10 transmit the counter value. This request is input to the processor 11 via the I/F circuit 13. The measurement instruction maybe, for example, a signal generated when an operation button provided in the measuring device 20 is pressed, or a signal automatically generated at a predetermined time interval or otherwise.

When the request from the MCU 21 is input, the processor 11 reads the counter value from the memory 16 and transmits the counter value to the measuring device 20 via the I/F circuit 13. Thus, the MCU 21 may obtain the counter value when the measurement value is acquired, that is, time information indicating when the measurement value was acquired.

The MCU 21 generates transmission data with the measurement value and the counter value when the measurement value was acquired, and outputs a signal that instructs wireless transmission to the processor 11 via the I/F circuit 13.

When the signal that instructs wireless transmission is sent from the MCU 21, the processor 11 (also referred to as a controller) adds the current counter value (present time information) when the transmission data is transmitted with the measurement value and the counter value when the measurement value was acquired, and outputs the current counter value and the transmission data to the transmission/reception circuit 14.

The transmission/reception circuit 14 transmits the transmission data including the measurement value, the counter value when the measurement value was acquired, and the current counter value as a beacon signal at a predetermined transmission interval via the antenna 17. In this way, the wireless IC 10 transmits the measurement value, the acquisition time for the measurement value, and the time information indicating when the measurement value was transmitted to the mobile terminal 30.

The mobile terminal 30 is, for example, a smartphone or a tablet, which wirelessly communicates with the wireless IC 10, and which receives the measurement result from the measuring device 20. The mobile terminal 30 such as a smartphone or a tablet generally has a clock function to determine the current time. Upon receiving the beacon signal from the wireless IC 10, the mobile terminal 30 calculates the time at which the measurement value was measured based on the time when the beacon signal was received, the counter value when the sensor 22 measured the measurement value, and the counter value when the beacon signal was output from the wireless IC 10.

Specifically, the mobile terminal 30 calculates a difference between the counter value for the measurement by the sensor 22 and the counter value when the beacon signal was output from the wireless IC 10 to determine how long it has been from when the measurement value was measured by the measuring device 20. As a result, the mobile terminal 30 may determine how long it has been from when the measurement value was measured from the time when the beacon signal was received, thereby calculating the time when the measurement value was measured. The mobile terminal 30 records the measurement value and the time corresponding to when the measurement value was measured in association with each other.

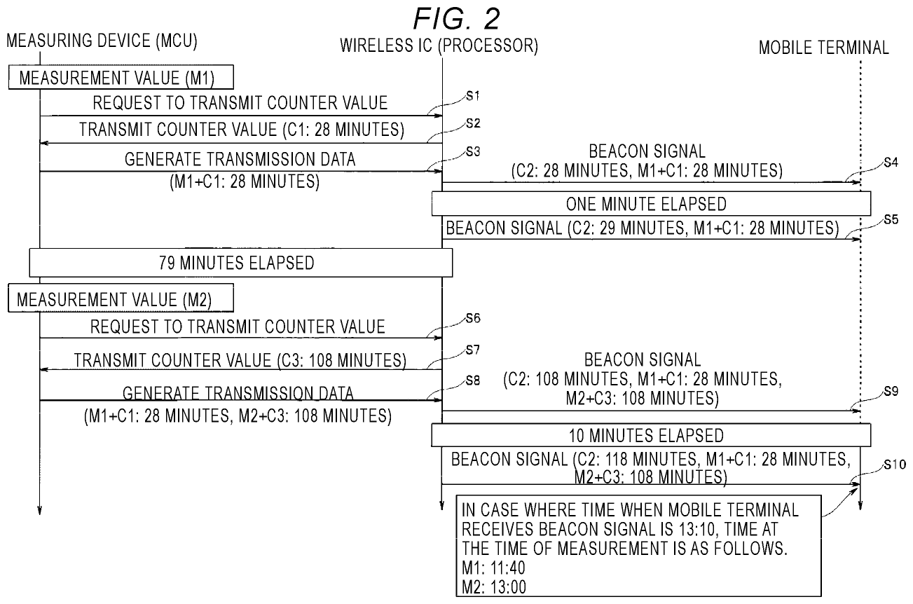

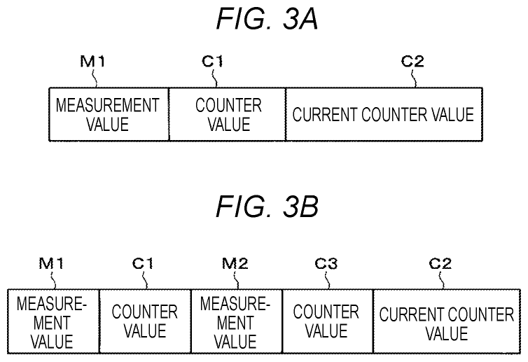

Next, the operation at the time of measurement by the measuring device 20 and data transmission by the wireless IC 10 will be described. FIG. 2 is a timing chart illustrating the operation at the time of measurement by the measuring device and data transmission by the wireless IC. FIGS. 3A and 3B are views illustrating an example of transmission data transmitted as a beacon signal.

First, when a measurement instruction is input to the measuring device 20, the MCU 21 of the measuring device 20 acquires a measurement value M1 from the sensor 22 and requests that the processor 11 transmit a counter value (S1). Upon receiving the request to transmit the counter value, the processor 11 of the wireless IC 10 reads a counter value C1 of the counter 15 out of the memory 16 and transmits the counter value C1 to the MCU 21 (S2).

The counter value C1 is a value corresponding to when the measurement value M1 is measured. More specifically, the counter value C1 is a value indicating the elapsed time from when the wireless IC 10 was powered on (or reset) until the measurement value M1 was measured. In the present embodiment, the counter value C1 indicates 28 minutes. That is, the measurement value M1 was measured 28 minutes after the wireless IC 10 was powered on (or reset signal).

The MCU 21 generates transmission data in which the counter value C1 is added to the measurement value M1, and instructs the processor 11 to perform a wireless transmission (S3). Upon receiving the wireless transmission instruction, the processor 11 adds the current counter value C2 to the transmission data (that is, the measurement value M1 and the counter value C1) and transmits the added current counter value C2 as a beacon signal (S4).

As illustrated in FIG. 3A, the beacon signal includes the measurement value M1, the counter value C1 when the measurement value M1 was measured, and the current counter value C2.

The counter value C2 is a value corresponding to when the beacon signal was transmitted. More specifically, the counter value C2 is a value indicating the elapsed time from when the wireless IC 10 was powered on or reset until the transmission data was transmitted. In the present embodiment, the counter value C2 indicates 28 minutes.

Thereafter, the processor 11 transmits the beacon signal at a predetermined interval while updating the current counter value C2. For example, when one minute has passed, the processor 11 adds a value indicating 29 minutes as the current counter value C2 and transmits the added value as the beacon signal (S5).

When a measurement instruction is input to the measuring device 20 after 79 minutes have elapsed from the process in S5, that is, after 108 minutes have elapsed after the wireless IC 10 was powered on or reset, the MCU 21 acquires a measurement value M2 from the sensor 22 and requests that the processor 11 transmit the counter value (S6). Upon being requested to transmit the counter value, the processor 11 reads a counter value C3 of the counter 15 from the memory 16 and transmits the counter value C3 to the measuring device 20 (S7).

The counter value C3 is a value when the measurement value M2 is measured, and indicates that the measurement value M2 is measured 108 minutes after the wireless IC 10 was powered on or reset.

The MCU 21 generates transmission data in which the second measurement value M2 and the counter value C3 when the measurement value M2 is measured are added to the first measurement value M1 and the counter value C1 when the measurement value M1 is measured, and instructs the processor 11 to perform a wireless transmission (S8).

Here, to account for the possibility that the mobile terminal 30 has not yet received the measurement value M1 and the counter value C1 measured the first time, the MCU 21 transmits the measurement value M1 and the counter value C1 measured the first time together with the measurement value M2 and the counter value C3 measured the second time.

The MCU 21 uses a plurality of measurement values M1 and M2 and a plurality of counter values C2 and C2 indicating when the measured values M1 and M2 were measured, as the transmission data. However, the present disclosure is not limited thereto and, in other examples, only the latest measured measurement value M2 and the counter value C2 indicating when the measurement value M2 was measured may be used as the transmission data.

Upon receiving the wireless transmission instruction, the processor 11 transmits the transmission data (measurement value M1+counter value C1 and measurement value M2+counter value C3) added with the current counter value C2 as the beacon signal (S9). In this way, the processor 11 transmits the transmission data including the plurality of measurement values and the plurality of counter values when the plurality of measurement values has been measured, and this is added to the current counter value.

As illustrated in FIG. 3B, the beacon signal includes the measurement value M1, the counter value C1 indicating when the measurement value M1 was measured, the measurement value M2, the counter value C3 indicating when the measurement value M2 was measured, and the current counter value C2.

Thereafter, the processor 11 transmits the beacon signal at a predetermined interval while updating the current counter value C2. For example, after 10 minutes have passed, the processor 11 adds a value indicating 118 minutes as the current counter value C2, and transmits the added value as the beacon signal (S10).

Here, it is assumed that the mobile terminal 30 has received the beacon signal transmitted by the process of S10. As described above, the mobile terminal 30 such as a smartphone generally has a clock function to determine the current time. The mobile terminal 30 is assumed to have received the beacon signal transmitted by the process of S10, for example, at 13:10 (1:10 p.m.).

In a case where the counter value for when the beacon signal is transmitted is 118 minutes and the counter value for when the measurement value M1 was measured is 28 minutes, the mobile terminal 30 may determine when (in clock time) the measurement value M1 was measured from the difference between the two counter values (90 minutes, in this example). Therefore, since the beacon signal is received at 13:10, the mobile terminal 30 calculates the time when the measurement value M1 was measured as being 11:40, which is 90 minutes prior to when the beacon signal was received. The mobile terminal 30 records the measurement value M1 and the time of measurement calculated for the measured value M1 in association with each other.

In a case where the counter value when the beacon signal was transmitted is 118 minutes and the counter value when the measurement value M2 was measured is 108 minutes, the mobile terminal 30 may determine the clock time at which the measurement value M2 was measured by the difference between the two counter values (10 minutes, in this case). Therefore, since the beacon signal was received at 13:10, the mobile terminal 30 calculates the time of measurement for the measurement value M2 as 13:00, which is ten minutes prior to when the beacon signal was received. The mobile terminal 30 records the measurement value M2 and the time of measurement calculated for measurement value M2 in association with each other.

In this way, when the wireless IC 10 transmits a measurement value measured by a measuring device 20 that does not have a clock function to the mobile terminal 30 by wireless communication, the wireless IC 10 transmits, to the mobile terminal 30, the measurement value, the counter value when the measurement value was measured/acquired, and the counter value when the measurement value was transmitted. As a result, the wireless IC 10 may supply the time information for the measurement value as measured by the measuring device 20 to the mobile terminal 30. This can be achieved, without adding a clock function to the measuring device 20 itself.

Therefore, according to the wireless IC of the present embodiment, it is possible to transmit the time information for the measurement value as measured by the measuring device to the transmission destination even when the measuring device does not have a clock function.

### Second Embodiment

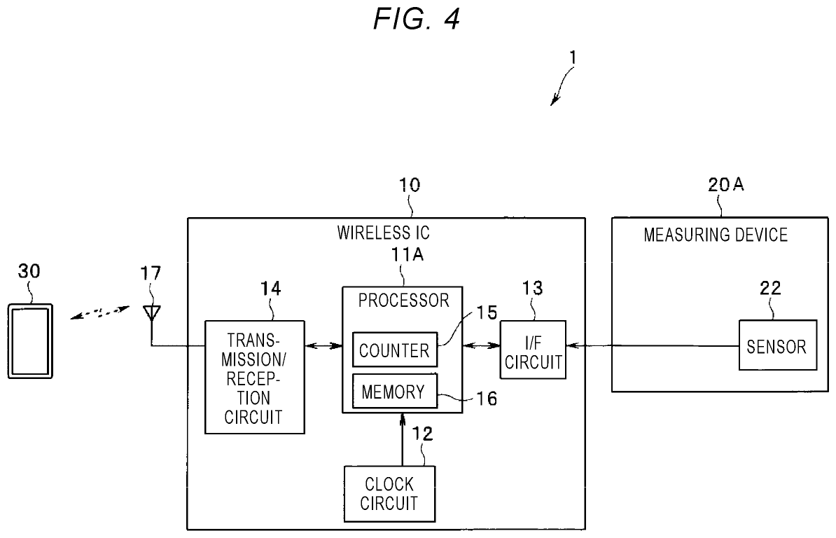

FIG. 4 is a block diagram illustrating the configuration of a wireless system including a wireless device according to the second embodiment. In FIG. 4, the same components as those in FIG. 1 are denoted by the same reference numerals, and an explanation thereof will not be repeated for sake of brevity.

As illustrated in FIG. 4, the wireless system 1 includes a measuring device 20A instead of the measuring device 20 of FIG. 1. Further, the wireless IC 10 includes a processor 11A instead of the processor 11 of FIG. 1.

In the measuring device 20A, the MCU 21 has been removed from the measuring device 20 of FIG. 1. That is, the measuring device 20A according to the present embodiment is configured to directly send a measurement value as measured by the sensor 22 to the processor 11A of the wireless IC 10.

When the measurement value measured by the sensor 22 is received, the processor 11A stores the measurement value and a counter value indicating when the measurement value was measured/received in the memory 16 in association with each other. Then, the processor 11A transmits, as a beacon signal, the measurement value, the counter value indicating when the measurement value is measured, and the current counter value by wireless communication.

Even if the measuring device 20A does not have an MCU, the processor 11A still transmits the measurement value along with the counter value indicating when the measurement value was measured and the counter value indicating when the measurement value was transmitted. As a result, in the same way as the first embodiment, the wireless IC of the second embodiment may transmit the time information for the measurement value measured by the measuring device to the transmission destination even when the measuring device does not have the clock function.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A wireless device, comprising:
a clock for generating clock cycles;
a wireless transmission/reception circuit;
a counter configured to count the number of clock cycles output by the clock since a previous point in time; and
a controller configured to append a first counter value from the counter to transmission data, the first counter value corresponding to a time of transmission for the transmission data from the wireless transmission/reception circuit, the transmission data further including a measurement value from a sensor device and a second counter value from the counter corresponding to a time of measurement for the measurement value.

2. The wireless device according to claim 1, wherein
the controller is configured to output the second counter value to the sensor device, and
the transmission data is received from the sensor device including the measurement value and the second counter value in association with each other.

3. The wireless device according to claim 1, wherein
when the measurement value is received from the measuring device, the controller is further configured to store the measurement value and the second counter value in a memory before the received transmission data is output from the wireless transmission/reception circuit.

4. The wireless device according to claim 1, wherein
the second counter value is based on an elapsed time from when the wireless device was last powered on or reset until the time of measurement for the measurement value, and
the first counter value is based on the elapsed time from when the wireless device was last powered on or reset until the time of transmission for the transmission data.

5. The wireless device according to claim 1, wherein
the controller is configured to cause the transmission/reception circuit to transmit transmission data including a plurality of measurement values measured by the sensor device and a plurality of second counter values each respectively corresponding to a time of measurement for the plurality of measurement values, the transmission data having the first count value appended thereto.

6. The wireless device according to claim 1, wherein the measurement value is received from the sensor device via Bluetooth Low Energy (BLE) communication.

7. A wireless system, comprising:
a sensor device configured to acquire measurement values; and
a wireless device including:
a clock for generating clock cycles;
a wireless transmission/reception circuit;
a counter configured to count the number of clock cycles output by the clock since a previous point in time; and
a controller configured to append a first counter value from the counter to transmission data, the first counter value corresponding to a time of transmission for the transmission data from the transmission/reception circuit, wherein
the transmission data further includes a measurement value from the sensor device and a second counter value from the counter corresponding to a time of measurement for the measurement value.

8. The wireless system according to claim 7, wherein the sensor device is a wearable device being worn by a person.

9. The wireless system according to claim 7, wherein
the controller is configured to output the second counter value to the sensor device, and
the transmission data is received from the sensor device including the measurement value and the second counter value in association with each other.

10. The wireless system according to claim 7, wherein when the measurement value is received from the measuring device, the controller is further configured to store the measurement value and the second counter value in a memory before the received transmission data is output from the wireless transmission/reception circuit.

11. The wireless system according to claim 7, wherein
the second counter value is based on an elapsed time from when the wireless device was last powered on or reset until the time of measurement for the measurement value, and
the first counter value is based on the elapsed time from when the wireless device was last powered on or reset until the time of transmission for the transmission data.

12. The wireless system according to claim 7, wherein the controller is configured to cause the transmission/reception circuit to transmit transmission data including a plurality of measurement values measured by the sensor device and a plurality of second counter values each respectively corresponding to a time of measurement for the plurality of measurement values, the transmission data having the first count value appended thereto.

13. The wireless system according to claim 12, wherein the measurement value is received from the sensor device via Bluetooth Low Energy (BLE) communication.

14. The wireless system according to claim 7, wherein the measurement value is received from the sensor device via Bluetooth Low Energy (BLE) communication.

15. A method of transmitting measurement data obtained from a sensor device without an internal clock, the method comprising:
counting a number of clock cycles output by a clock in a wireless device to provide a counter value corresponding to the number of clock cycles since a previous point in time;
transmitting a measurement counter value to the sensor device;
receiving a measurement value from the sensor device in conjunction with the previously transmitted measurement counter value; and
associating the measurement value and the measurement counter value with a current counter value and then transmitting the measurement value, the measurement counter value, and the associated current counter value to an external device.

16. The method according to claim 15, wherein the measurement value is received from the sensor device via Bluetooth Low Energy (BLE) communication.

17. The method according to claim 15, further comprising:
receiving a request from the sensor device for the transmission of the measurement counter value to the sensor device before transmitting the measurement counter value to the sensor device.

18. The method according to claim 15, wherein
the sensor device is a wearable device, and
the measurement values are biological information from a person wearing the sensor device.

19. The method according to claim 15, wherein the sensor device is a sphygmomanometer.

20. The method according to claim 15, wherein a plurality of measurement values measured by the sensor device, and respectively received in conjunction with a plurality of measurement counter values, each respectively corresponding to a time of measurement for the plurality of measurement values is associated with just one current counter value before transmission to the external device.
